(19) United States
(12) Patent Application Publication
(10) Pub. No.: US 2026/0033729 A1
(43) Pub. Date: Feb. 5, 2026

(54) PHYSIOLOGICAL INFORMATION DISPLAY APPARATUS AND PHYSIOLOGICAL INFORMATION DISPLAY METHOD

(71) Applicant: NIHON KOHDDEN CORPORATION, Tokyo (JP)

(72) Inventors: Yoshiharu HARADA, Tokorozawa-shi, Saitama (JP); Yoshihiro SUGO, Tokorozawa-shi, Saitama (JP); Sayaka YAMAZAKI, Tokorozawa-shi, Saitama (JP); Tomoyuki SAKAI, Tokorozawa-shi, Saitama (JP)

(73) Assignee: NIHON KOHDEN CORPORATION, Tokyo (JP)

(21) Appl. No.: 19/101,450

(22) PCT Filed: Aug. 4, 2023
(86) PCT No.: PCT/JP2023/028588

§ 371 Date: Feb. 5, 2025

(30) Foreign Application Priority Data
Aug. 10, 2022 (JP) 2022-127788

(51) Int. Cl. A61B 5/0205; A61B 5/00; A61B 5/022; A61B 5/024; A61B 5/08

(52) U.S. Cl. CPC A61B 5/0205; A61B 5/7242; A61B 5/742; A61B 5/022; A61B 5/024; A61B 5/0816

(57) ABSTRACT

A physiological information display apparatus includes a heart rate acquisition unit configured to acquire a heart rate of a subject, a respiration rate acquisition unit configured to acquire a respiration rate of the subject, a hemodynamics calculation unit configured to calculate a hemodynamic parameter of the subject, and a respiratory variation of the hemodynamic parameter, based on a plurality of the hemodynamic parameters calculated in a predetermined period, and a display controller configured to output the respiratory variation to a display. The hemodynamics calculation unit is configured to calculate a heart rate in one respiration. The display controller is configured to output a setting screen for setting a length of the predetermined period. The hemodynamics calculation unit is configured to acquire setting information, set the length of the predetermined period based on the acquired setting information; and calculate the respiratory variation in the set predetermined period.

### TECHNICAL FIELD

The present disclosure relates to a physiological information display apparatus and a physiological information display method.

In the related art, when performing a treatment such as administration of a fluid to a subject, as a useful parameter for checking a state of the subject, a change in a parameter related to hemodynamics (hereinafter, referred to as a “hemodynamic parameter”) such as a respiratory variation of stroke volume (hereinafter, also referred to as a “stroke volume variation (SVV)”) of the subject or a respiratory variation of pulse pressure (hereinafter, also referred to as a “pulse pressure variation (PPV)”) is used.

Specifically, when an intrathoracic pressure varies due to respiration, the blood is pushed out, and the stroke volume or the pulse pressure varies. In a state where a total blood volume is small, fluctuation of the SVV or PPV is large, and thus a large value of SVV or PPV indicates that the total blood volume is insufficient. JP-A-2011-55961 discloses a method for reducing artifact in an apparatus for analyzing SVV.

### NON-PATENT LITERATURE

Non-Patent Literature 1: Daniel De Backer, Fabio Silvio Taccone, Roland Holsten, Fayssal Ibrahimi, Jean-Louis Vincent: Influence of Respiratory Rate on Stroke Volume Variation in Mechanically Ventilated Patients, Anesthesiology, issued in May 2009, Volume 110, Issue 5, Pages 1092-1097

### SUMMARY OF INVENTION

### Technical Problem

In the apparatus disclosed in JP-A-2011-55961, the SVV for one respiratory cycle is calculated. However, a heart rate in one respiratory cycle may be low. In such a case, the number of pieces of data of the stroke volume used for calculating the SVV is not sufficient, and a technique capable of more accurately monitoring a state of a subject is desired.

The present disclosure provides a physiological information display apparatus and a physiological information display method capable of more accurately monitoring a state of a subject.

### Solution to Problem

A first aspect of the disclosure of a physiological information display apparatus includes:
a heart rate acquisition unit configured to acquire a heart rate of a subject;
a respiration rate acquisition unit configured to acquire a respiration rate of the subject;
a hemodynamics calculation unit configured to calculate:
a hemodynamic parameter of the subject; and
a respiratory variation of the hemodynamic parameter, based on a plurality of the
hemodynamic parameters calculated in a predetermined period; and
a display controller configured to output the respiratory variation to a display,
in which the hemodynamics calculation unit is further configured to calculate a heart rate in one respiration, using the heart rate and the respiration rate, the heart rate in one respiration being a heart rate included in one respiratory cycle,
the display controller is further configured to output, to the display, a setting screen for setting a length of the predetermined period, the setting screen configured to display the heart rate in one respiration, and
the hemodynamics calculation unit is configured to:
acquire setting information indicating contents set on the setting screen;
set the length of the predetermined period based on the acquired setting information; and calculate the respiratory variation in the set predetermined period.

A second aspect of the disclosure of a physiological information display method includes:
acquiring a heart rate of a subject;
acquiring a respiration rate of the subject;
calculating a heart rate in one respiration using the heart rate and the respiration rate, the heart rate in one respiration being a heart rate included in one respiratory cycle;
calculating a hemodynamic parameter of the subject;
outputting, to a display, a setting screen for setting a length of a predetermined period, the setting screen configured to display the heart rate in one respiration;
setting the length of the predetermined period based on contents set on the setting screen; calculating a respiratory variation of the hemodynamic parameter, based on a plurality of the hemodynamic parameters calculated in the set predetermined period; and outputting the respiratory variation to the display.

### Advantageous Effects of Invention

According to the present disclosure, it is possible to more accurately monitor a state of a subject.

### BRIEF DESCRIPTION OF DRAWINGS

FIG. 1 illustrates a configuration of a physiological information processing apparatus according to an aspect of the present disclosure.

FIG. 2 illustrates an example of a measurement mode using a monitor device that is an example of the physiological information processing apparatus illustrated in FIG. 1.

FIG. 3 illustrates a configuration of a hemodynamics calculation unit of FIG. 1.

FIG. 4 illustrates an example of a screen displayed on a display of FIG. 1.

FIG. 5 is a diagram for explaining that the number of pieces of used data used to calculate a respiratory variation changes according to the number of cycles set on a setting screen illustrated in FIG. 4.

FIG. 6 is a diagram for explaining a state where a length of a predetermined period is manually changed by an operator using a period setting unit illustrated in FIG. 3.

FIG. 7 is a diagram for explaining a state where a length of a predetermined period is automatically changed by the period setting unit illustrated in FIG. 3.

FIG. 8 is a diagram for explaining a state where a length of a predetermined period is automatically changed by the period setting unit illustrated in FIG. 3.

FIG. 9 is a diagram for explaining a state where a length of a predetermined period is automatically changed by the period setting unit illustrated in FIG. 3.

FIG. 10 is a flowchart for explaining an outline of operations performed when calculating and displaying a respiratory variation of a hemodynamic parameter by the physiological information processing apparatus according to the embodiment of the present disclosure.

FIG. 11 is a flowchart for explaining operations in a case where the hemodynamics calculation unit illustrated in FIG. 1 operates in a manual mode.

FIG. 12 is a flowchart for explaining operations in a case where the hemodynamics calculation unit illustrated in FIG. 1 operates in an automatic mode.

### DESCRIPTION OF EMBODIMENTS

Exemplary embodiments of a physiological information display apparatus and a physiological information display method according to a presently disclosure will be described below with reference to the accompanying drawings.

### <Configuration of Physiological Information Processing Apparatus>

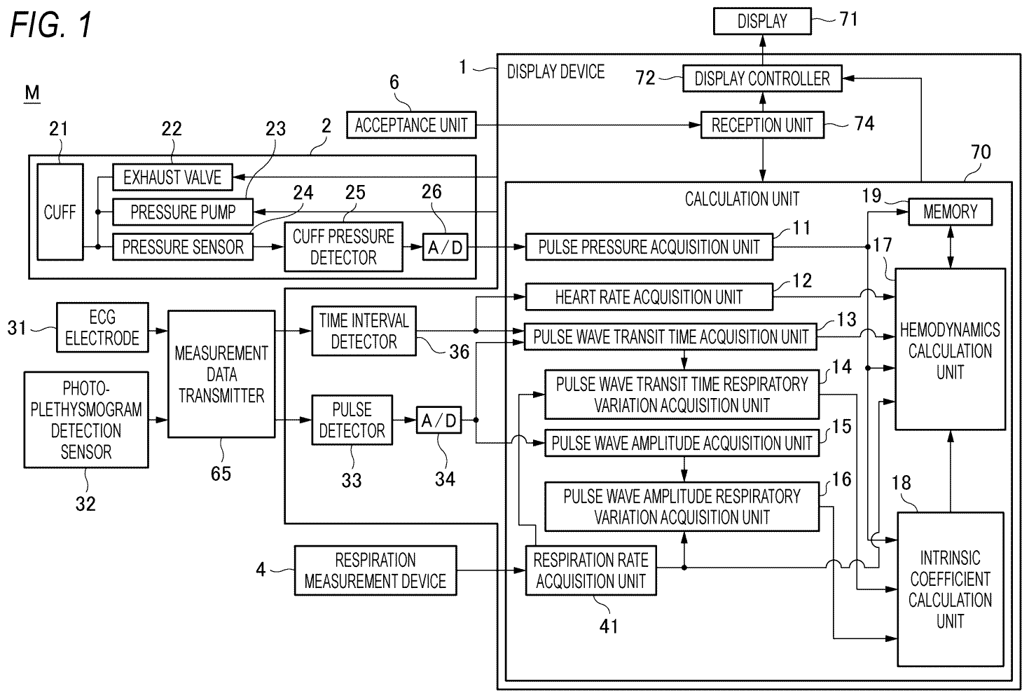

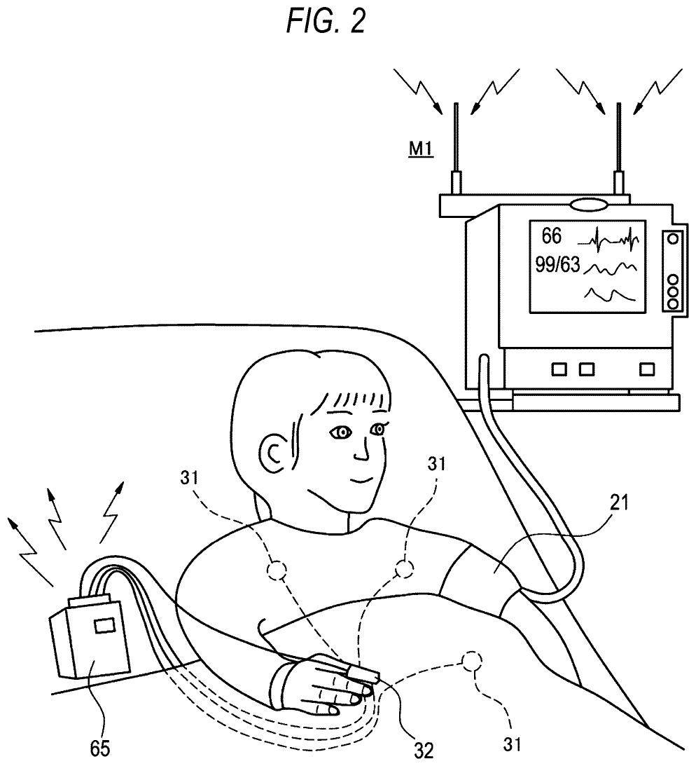

FIG. 1 illustrates a configuration of a physiological information processing apparatus (physiological information display apparatus) M according to an aspect of the present disclosure. FIG. 2 illustrates an example of a measurement mode using a monitor device M1 that is an example of the physiological information processing apparatus M illustrated in FIG. 1.

The physiological information processing apparatus M may include a display device 1 configured to perform calculation, display control, and the like of a hemodynamic parameter related to hemodynamics of a subject, a blood pressure measurement device 2 configured to measure a systolic blood pressure and a diastolic blood pressure of the heart, a respiration measurement device 4, an acceptance unit 6, an ECG electrode 31, a photoplethysmogram detection sensor 32, a measurement data transmitter 65, and a display 71.

The blood pressure measurement device 2 is designed to configured to measure a blood pressure of a subject by using a noninvasive blood pressure (NIBP) measurement method. The blood pressure measurement device 2 may include a cuff 21, an exhaust valve 22, a pressure pump 23, a pressure sensor 24, a cuff pressure detector 25, and an A/D converter 26. Specifically, as illustrated in FIG. 2, the blood pressure measurement device 2 is configured to measure a blood pressure with the cuff 21 attached to the upper arm of a subject.

An interior of the cuff 21 is configured to be opened or closed to the atmosphere by opening and closing the exhaust valve 22. The exhaust valve 22 is configured to be opened and closed based on a control signal output from the display device 1. The pressure pump 23 is configured to supply air to the cuff 21. The supply of air is controlled based on a control signal output from the display device 1.

The pressure sensor 24 is connected to the cuff 21. A sensor output of the pressure sensor 24 is detected by the cuff pressure detector 25. A sensor output from the cuff pressure detector 25 is converted into a digital signal by the A/D converter 26 and then is input to a pulse pressure acquisition unit 11 of the display device 1.

As illustrated in FIG. 2, the ECG electrode 31 is attached to the chest of the subject, and is configured to perform measurement with an R wave generation time point in an electrocardiogram as a reference point of a time interval. The ECG electrode 31 is electrically connected to the measurement data transmitter 65. Measurement data from the ECG electrode 31 is input to the measurement data transmitter 65 and then is wirelessly transmitted from the measurement data transmitter 65 to a time interval detector 36 in the display device 1 illustrated in FIG. 1.

As illustrated in FIG. 2, the photoplethysmogram detection sensor 32 is attached to a subject's periphery such as a finger, and is configured to measure, for example, pulse wave. Pulse wave transit time (PWTT) is obtained based on the measurement data obtained from the ECG electrode 31 and the pulse wave obtained from the photoplethysmogram detection sensor 32. The photoplethysmogram detection sensor 32 is electrically connected to the measurement data transmitter 65. Measurement data obtained by the photoplethysmogram detection sensor 32 is input to the measurement data transmitter 65, and is wirelessly transmitted from the measurement data transmitter 65 to a pulse detector 33 in the display device 1 illustrated in FIG. 1.

The respiration measurement device 4 is configured to continuously measure respiration of the subject. Measurement data obtained by measurement of the respiration measurement device 4 is input to a respiration rate acquisition unit 41 of the display device 1.

The acceptance unit 6 is configured to accept an input operation of the operator and is configured to generate an instruction signal corresponding to the input operation. The acceptance unit 6 is, for example, a touch panel disposed overlapping the display 71 described later, an operation button provided on a housing of the display device 1, or a mouse or keyboard connected to an input and output interface that is not illustrated (for example, a USB interface). The instruction signal generated by the acceptance unit 6 is input to the display device 1.

### <Configuration of Display Device>

The display device 1 may include the pulse detector 33, an A/D converter 34, the time interval detector 36, a calculation unit 70, a display controller 72, and a reception unit 74. The calculation unit 70 may include the pulse pressure acquisition unit 11, a heart rate acquisition unit 12, a pulse wave transit time acquisition unit 13 (hereinafter, PWTT acquisition unit 13), a pulse wave transit time respiratory variation acquisition unit 14 (hereinafter, PWTTRV acquisition unit 14), a pulse wave amplitude acquisition unit 15 (hereinafter, PWA acquisition unit 15), a pulse wave amplitude respiratory variation acquisition unit 16 (hereinafter, PWARV acquisition unit 16), a hemodynamics calculation unit 17, an intrinsic coefficient calculation unit 18, a memory 19, and the respiration rate acquisition unit 41. The display device 1 may include one or more central processing unit (CPU), a read only memory (ROM), a random access memory (RAM), a hard disk drive (HDD), and the like. The CPU may function as the calculation unit 70, the display controller 72, and the reception unit 74 and the like.

The time interval detector 36 is configured to acquire an ECG waveform based on the measurement data that is received from the ECG electrode 31 via the measurement data transmitter 65. The time interval detector 36 is configured to convert the measurement data into a digital signal and is configured to output the digital signal to the heart rate acquisition unit 12 and the PWTT acquisition unit 13 of the calculation unit 70.

The pulse detector 33 is configured to acquire a waveform of the photoplethysmogram in the periphery, based on the measurement data that is received from the photoplethysmogram detection sensor 32 via the measurement data transmitter 65. Then, the pulse detector 33 is configured to output the measurement data to the A/D converter 34. The A/D converter 34 is configured to convert the measurement data into a digital signal and is configured to output the digital signal to the PWTT acquisition unit 13 and the pulse wave amplitude acquisition unit 15 of the calculation unit 70.

The pulse pressure acquisition unit 11 is configured to measure a pulse pressure (PP) of the subject, based on blood pressure data obtained by measurement of the blood pressure measurement device 2. The pulse pressure PP is calculated, for example, from a difference between a systolic (maximum) blood pressure value and a diastolic (minimum) blood pressure value. The measured pulse pressure PP is input to the intrinsic coefficient calculation unit 18. The pulse pressure acquisition unit 11 is configured to store, in the memory 19, for example, the pulse pressure PP and a current time-point in association with each other.

The heart rate acquisition unit 12 is configured to calculate the number of beats in one minute (heart rate, HR), based on the reference point (R wave generation time-point) measured by the time interval detector 36. The calculated heart rate HR is input to the hemodynamics calculation unit 17.

The PWTT acquisition unit 13 is configured to calculate the PWTT, which is time lapsed from occurrence of the R wave to occurrence of a SpO2 pulse wave in the electrocardiogram, based on the reference point (R wave generation time-point) measured by the time interval detector 36 and based on the waveform in the periphery detected by the photoplethysmogram detection sensor 32. The PWTT acquisition unit 13 is configured to output the calculated PWTT to the hemodynamics calculation unit 17 and to the PWTTRV acquisition unit 14.

The respiration rate acquisition unit 41 is configured to detect a respiratory cycle of the subject, based on respiration data measured by the respiration measurement device 4. For example, the respiration measurement device 4 is a detector configured to detect a concentration of carbon dioxide in expired air. The respiration rate acquisition unit 41 is configured to specify at least one of a start timing and an end timing of the respiration of the subject, based on a detection result of the respiration measurement device 4. The respiration rate acquisition unit 41 is configured to detect the respiratory cycle of the subject, based on the specified timing.

In addition, the respiration rate acquisition unit 41 is configured to detect a respiration rate (RR) per minute of the subject, based on the respiration data. Then, the respiration rate acquisition unit 41 is configured to input the detected respiratory cycle and respiratory rate RR to the PWTTRV acquisition unit 14, to the pulse wave amplitude respiratory variation acquisition unit 16, and to the hemodynamics calculation unit 17. For example, the respiration rate acquisition unit 41 is configured to notify, as a notification of the respiratory cycle, at least one of the start timing and the end timing of the respiratory cycle.

The respiration measurement device 4 is not limited to a device configured to detect the concentration of carbon dioxide in the expired air, and may be a device configured to measure the respiration of the subject by using a change in impedance, a device configured to measure an anesthetic gas output from an anesthesia apparatus, or the like.

The physiological information processing apparatus M may not include the respiration measurement device 4. In this case, for example, the physiological information processing apparatus M sets a length of one respiratory cycle to a fixed value such as 20 seconds, and is configured to determine at least one of the start timing and the end timing of the respiratory cycle.

Based on the PWTT calculated by the PWTT acquisition unit 13 and the respiratory cycle detected by the respiration rate acquisition unit 41, the PWTTRV acquisition unit 14 is configured to measure the respiratory variation of the PWTT. Measurement data indicating the measured respiratory variation of the PWTT is input to the intrinsic coefficient calculation unit 18.

The pulse wave amplitude acquisition unit 15 is configured to measure an amplitude of a pulse wave from the waveform of the periphery acquired by the pulse detector 33. The measured pulse wave amplitude is input to the pulse wave amplitude respiratory variation acquisition unit 16.

Based on the pulse wave amplitude measured by the pulse wave amplitude acquisition unit 15 and the respiratory cycle detected by the respiration rate acquisition unit 41, the pulse wave amplitude respiratory variation acquisition unit 16 is configured to measure a respiratory variation of the pulse wave amplitude (pulse amplitude variation, PAV). The measured pulse amplitude variation PAV is input to the intrinsic coefficient calculation unit 18.

Based on the pulse pressure PP measured by the pulse pressure acquisition unit 11, based on the respiratory variation of the PWTT measured by the PWTTRV acquisition unit 14, and based on the pulse amplitude variation PAV measured by the pulse wave amplitude respiratory variation acquisition unit 16, the intrinsic coefficient calculation unit 18 is configured to calculate coefficients intrinsic to the subject. The calculated coefficients are, for example, coefficients K, α, or β to be described later, and are input to the hemodynamics calculation unit 17.

<Configuration of Hemodynamics Calculation Unit>

### (Calculation of Respiratory Variation of Hemodynamic Parameter)

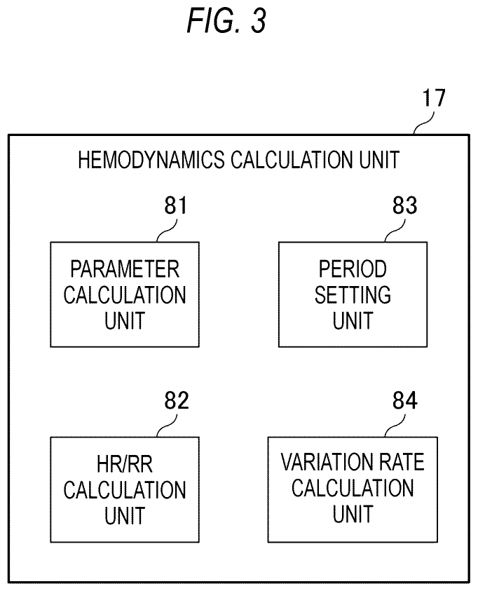

FIG. 3 illustrates a configuration of the hemodynamics calculation unit 17 of FIG. 1. As illustrated in FIG. 3, the hemodynamics calculation unit 17 may include a parameter calculation unit 81, an HR/RR calculation unit 82, a period setting unit 83, and a variation rate calculation unit 84.

The parameter calculation unit 81 is configured to calculate a hemodynamic parameter of the subject, based on the heart rate HR calculated by the heart rate acquisition unit 12, based on the PWTT measured by the PWTT acquisition unit 13, and based on the coefficients K, α, and β calculated by the intrinsic coefficient calculation unit 18. The parameter calculation unit 81 is configured to calculate, as a hemodynamic parameter, for example, a flow rate (stroke volume, SV) of the blood flowing into the aorta during systole of the heart.

There is a correlation between stroke volume SV and PWTT as shown in Equation 1. In Equation 1, K, α, and β are coefficients intrinsic to the subject.

$$SV = K * ( α * PWTT + β ) \qquad ( Equation 1 )$$

The parameter calculation unit 81 is configured to substitute, into Equation 1, the coefficients K, α, and β calculated by the intrinsic coefficient calculation unit 18. The parameter calculation unit 81 is configured to substitute, into the PWTT in Equation 1, for example, the PWTT received from the PWTT acquisition unit 13. Accordingly, the parameter calculation unit 81 can calculate the stroke volume SV. The stroke volume SV calculated by the parameter calculation unit 81 is hereinafter referred to as “stroke volume esSV”.

For example, the parameter calculation unit 81 is configured to periodically calculate the stroke volume esSV, is configured to associate the calculated stroke volume esSV with a calculation timing of the stroke volume esSV, and is configured to store the calculated stroke volume esSV and the calculation timing in the memory 19 illustrated in FIG. 1.

For example, when receiving an input of a newly detected respiratory cycle from the respiration rate acquisition unit 41, the variation rate calculation unit 84 is configured to calculate a respiratory variation of the hemodynamic parameter in a predetermined period based on the respiratory cycle. The predetermined period is the length of the most recent one or more respiratory cycles.

Here, it is assumed that the predetermined period is the most recent one respiratory cycle detected by the respiration rate acquisition unit 41. In this case, the variation rate calculation unit 84 is configured to read out a plurality of stroke volumes esSV calculated in the most recent one respiratory cycle from among a plurality of stroke volumes esSV stored in the memory 19.

Then, the variation rate calculation unit 84 is configured to specify a maximum value esSVmax in the read-out stroke volumes esSV and to specify a minimum value esSVmin in the read-out stroke volumes esSV. Then, by using the maximum value esSVmax and the minimum value esSVmin, the variation rate calculation unit 84 is configured to calculate a respiratory variation of the stroke volume esSV (that is, a stroke volume variation, SVV) as indicated by the following Equation 2. The variation rate calculation unit 84 is configured to store the calculated respiratory variation SVV in the memory 19.

$$SVV = 2 * ( esSV max - esSV min ) / ( esSV max + esSV min ) \qquad ( Equation 2 )$$

As the respiratory variation of the hemodynamic parameter in a predetermined period, the variation rate calculation unit 84 may calculate a respiratory variation of the pulse pressure PP (that is, a pulse pressure variation, PPV) measured by the pulse pressure acquisition unit 11 illustrated in FIG. 1 instead of the stroke volume variation SVV.

For example, when receiving an input of a newly detected respiratory cycle from the respiration rate acquisition unit 41, the variation rate calculation unit 84 is configured to read out a plurality of pulse pressures PP measured in the most recent one respiratory cycle among a plurality of pulse pressures PP stored in the memory 19.

Then, the variation rate calculation unit 84 is configured to specify a maximum value PPmax in the read-out pulse pressures PP and to specify a minimum value PPmin in the read-out pulse pressures PP. By using the maximum value PPmax and the minimum value PPmin, the variation rate calculation unit 84 is configured to calculate the pulse pressure variation PPV as in the following Equation 3. The variation rate calculation unit 84 is configured to store the calculated respiratory variation PPV in the memory 19.

$$PPV = 2 * ( PP max - PP min ) / ( PP max + PP min ) \qquad ( Equation 3 )$$

### (Display of Respiratory Variation)

The display controller 72 is configured to output, to the display 71 such as a monitor, the hemodynamic parameter and the respiratory variation of the hemodynamic parameter, which are calculated by the variation rate calculation unit 84. Accordingly, a screen including a screen configured to display the hemodynamic parameter and the respiratory variation of the hemodynamic parameter is displayed on the display 71.

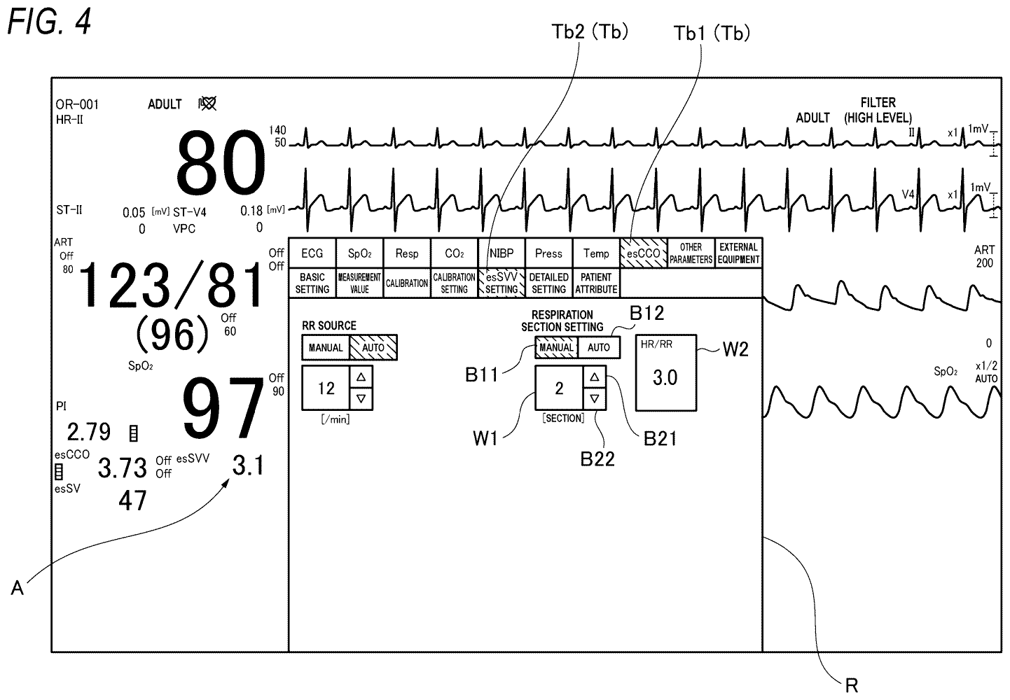

FIG. 4 illustrates an example of a screen displayed on the display 71 of FIG. 1. As illustrated in FIG. 4, on the screen displayed on the display 71, the latest heart rate HR, blood pressure value, noninvasive estimated continuous cardiac output esCCO, stroke volume esSV, and the like of the subject are displayed. As will be described later, the screen may include a region R for performing setting related to a hemodynamic parameter. The region R is displayed, for example, in a case where a menu button (not illustrated) displayed on the screen is selected by the operator.

For example, the display controller 72 is configured to periodically refer to the memory 19 and configured to perform control such that the latest hemodynamic parameter is displayed on the screen. In the example illustrated in FIG. 4, a value “3.73” of the noninvasive estimated continuous cardiac output esCCO and a value “47” of the stroke volume esSV are displayed as examples of the latest hemodynamic parameters.

For example, the display controller 72 is configured to periodically refer to the memory 19 and configured to perform control such that a respiratory variation of the latest hemodynamic parameter is displayed on the screen. In the example illustrated in FIG. 4, as indicated by an arrow A, a value “3.1” of the respiratory variation SVV is displayed as an example of a respiratory variation of the latest hemodynamic parameter.

### (Setting of Predetermined Period)

Here, in a case where the predetermined period is the most recent one respiratory cycle, the heart rate in the predetermined period may be small. In such a case, there is a problem that the number of pieces of data of the hemodynamic parameter used to calculate the respiratory variation (hereinafter, referred to as “the number of pieces of used data”) is not sufficient.

Specifically, Non-Patent Literature 1 discloses that a value (HR/RR) obtained by dividing the heart rate HR by the respiration rate RR is larger than 3.6, as a condition for detecting a respiratory variation of a hemodynamic parameter. The value of HR/RR corresponds to a heart rate included in one respiratory cycle. Hereinafter, HR/RR is also referred to as a “heart rate in one respiration”. Generally, in a healthy adult, the heart rate HR is about 80, the respiration rate RR is about 12, and HR/RR, which is the heart rate in one respiration, is about 6.7.

In a case where HR/RR, which is the heart rate in one respiration, is 3.6 or less since the heart rate HR is low or the respiration rate RR is high, the number of pieces of data of the hemodynamic parameter calculated in one respiratory cycle, that is, the number of pieces of used data is insufficient. Therefore, the hemodynamics calculation unit 17 illustrated in FIG. 3 can change a length of the predetermined period used to calculate the respiratory variations SVV and PPV.

### (a) Display of Setting Screen

As illustrated in FIG. 4, the region R may include a plurality of tabs Tb. The plurality of tabs Tb may include, for example, a tab Tb1 for selecting a display related to the hemodynamic parameter and a tab Tb2 for selecting a display of a setting screen of a predetermined period. For example, letters “esCCO” are assigned to the tab Tb1. For example, letters “esSVV setting” are assigned to the tab Tb2. FIG. 4 illustrates a screen displayed in a case where the operator performs, on the acceptance unit 6 illustrated in FIG. 1, an input operation of selecting the tab Tb1 and further selecting the tab Tb2.

In a case where the input operation as described above is performed, the acceptance unit 6 outputs an instruction signal indicating contents of the input operation to the reception unit 74 of the display device 1. Upon receiving the instruction signal output from the acceptance unit 6, the reception unit 74 outputs the instruction signal to the display controller 72. Upon receiving the instruction signal output from the reception unit 74, the display controller 72 performs control such that a setting screen as shown in FIG. 4 is displayed in the region R.

The setting screen may include a selection button B1I for selecting to manually change the length of the predetermined period, a selection button B12 for selecting to automatically change the length of the predetermined period, a window W1 in which the number of respiratory cycles included in the predetermined period (hereinafter, also simply referred to as “the number of cycles”) is displayed, a window W2 in which the value of the heart rate in one respiration HR/RR is displayed, a selection button B21 for selecting to increase the number of cycles, and a selection button B22 for selecting to decrease the number of cycles.

For example, the HR/RR calculation unit 82 illustrated in FIG. 3 is configured to periodically calculate the heart rate in one respiration HR/RR by using the heart rate HR calculated by the heart rate acquisition unit 12 and the respiration rate RR detected by the respiration rate acquisition unit 41. Then, the HR/RR calculation unit 82 is configured to store the calculated heart rate in one respiration HR/RR in the memory 19. The display controller 72 is configured to perform control of displaying, in the window W2, the latest heart rate in one respiration HR/RR stored in the memory 19.

The operator can select one of the selection buttons B1I and B12. In a state where neither the selection button B11 nor the selection button B12 is selected by the operator, the selection button B11 is automatically selected. That is, in such a state, the hemodynamics calculation unit 17 is configured to operate in a manual mode to be described later, and does not change the length of the predetermined period in a case where no operation of the operator is received.

The operator can select the selection button B21 and the selection button B22. For example, it is assumed that the operator performs an operation of selecting the selection button B21. In this case, the acceptance unit 6 illustrated in FIG. 1 outputs an instruction signal (setting information) indicating contents of the operation to the reception unit 74. Upon receiving the instruction signal output from the acceptance unit 6, the reception unit 74 outputs the instruction signal to the display controller 72 and the calculation unit 70. Upon receiving the instruction signal output from the reception unit 74, the display controller 72 increases a numeral displayed in the window W1 by one.

In the calculation unit 70, the period setting unit 83 illustrated in FIG. 3 is configured to set the length of the predetermined period, based on the respiratory cycle detected by the respiration rate acquisition unit 41 and the number of cycles set on the setting screen. More specifically, in a case where the period setting unit 83 receives, from the reception unit 74, the instruction signal indicating that the selection button B21 is selected, the period setting unit 83 resets the length of the predetermined period by increasing the number of cycles by one based on the instruction signal so that a period corresponding to the number of cycles after increase becomes the predetermined period. Then, the period setting unit 83 is configured to notify the variation rate calculation unit 84 of the reset predetermined period.

For example, it is assumed that the operator performs an operation of selecting the selection button B22 with respect to the acceptance unit 6. In this case, the acceptance unit 6 outputs an instruction signal indicating contents of the operation to the reception unit 74. Upon receiving the instruction signal output from the acceptance unit 6, the reception unit 74 outputs the instruction signal to the display controller 72 and the calculation unit 70. Upon receiving the instruction signal output from the reception unit 74, the display controller 72 decreases the numeral displayed in the window W1 by one.

In a case where the period setting unit 83 of the calculation unit 70 receives, from the reception unit 74, the instruction signal indicating that the selection button B22 is selected, the period setting unit 83 resets the length of the predetermined period by decreasing the number of cycles by one based on the instruction signal so that a period corresponding to the number of cycles after decrease becomes the predetermined period. Then, the period setting unit 83 is configured to notify the variation rate calculation unit 84 of the reset predetermined period.

Based on the predetermined period notified from the period setting unit 83, the variation rate calculation unit 84 is configured to calculate the respiratory variations SVV and PPV of the hemodynamic parameters, using a plurality of hemodynamic parameters calculated in the predetermined period.

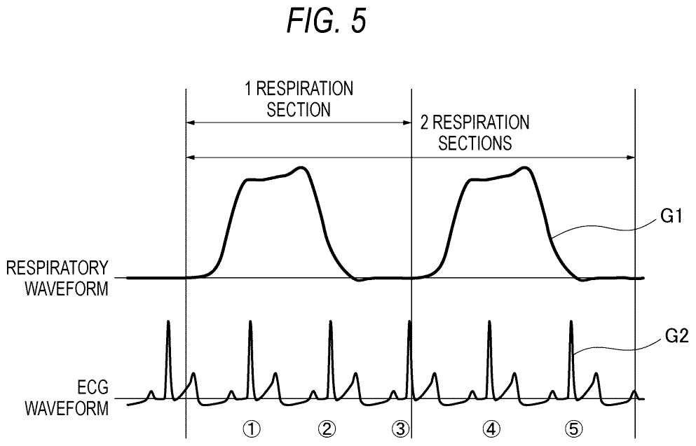

FIG. 5 is a diagram illustrating that the number of pieces of used data used to calculate the respiratory variations SVV and PPV changes according to the number of cycles set on the setting screen illustrate in FIG. 4. In FIG. 5, a respiratory waveform based on the measurement data obtained by measurement of the respiration measurement device 4 illustrated in FIG. 1 is shown by a graph G1, and an ECG waveform based on the measurement data obtained by the ECG electrode 31 illustrated in FIG. 1 is shown by a graph G2.

In a state where the respiratory waveform shown in the graph G1 and the ECG waveform shown in the graph G2 are obtained, in a case where the number of cycles included in the predetermined period is “1”, the heart rate included in the predetermined period is “3”. In a case where the number of cycles included in the predetermined period is “2”, the heart rate included in the predetermined period is “5”. As described above, as the number of cycles increases, the heart rate increases, and the number of pieces of used data used to calculate the respiratory variations SVV and PPV increases.

### (b) Manual Mode

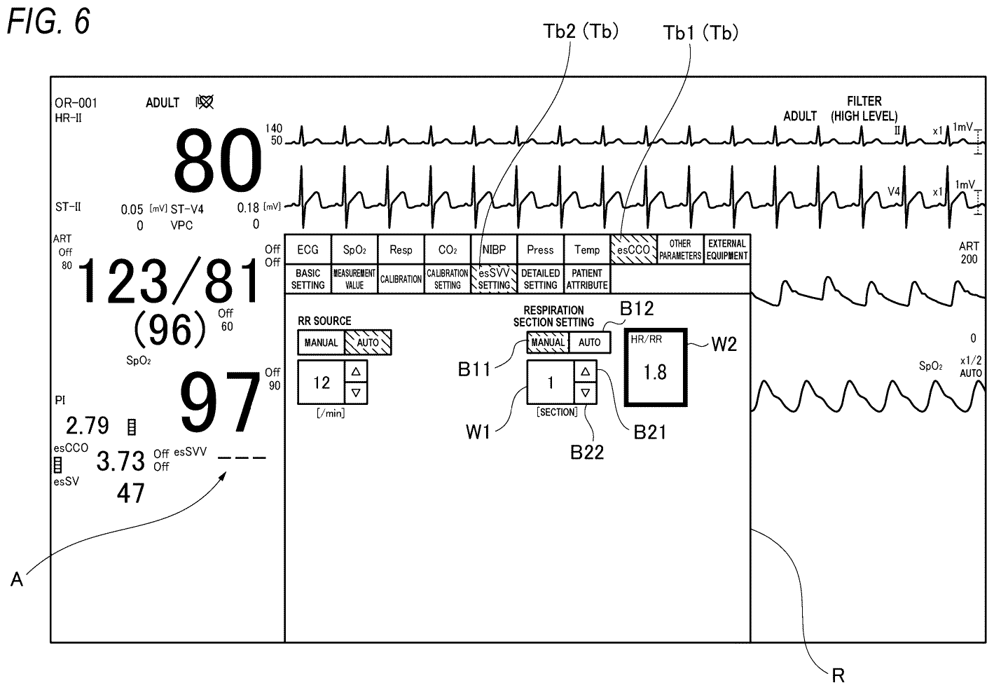

FIG. 6 is a diagram illustrating a state where change of the length of the predetermined period to be executed by the period setting unit 83 is manually performed by the operator illustrated in FIG. 3. Here, a case where the stroke volume variation SVV is calculated and displayed as a respiratory variation of the hemodynamic parameter will be described.

In FIG. 6, same as or similarly to FIG. 4, a setting screen is displayed in the region R. It is also assumed that the selection button B11 is selected on the setting screen. In this case, the hemodynamics calculation unit 17 operates in the manual mode.

As illustrated in FIG. 6, it is assumed that “1” is displayed in the window W1 and “1.8” is displayed in the window W2. In the following description, a threshold of the heart rate in one respiration HR/RR is set to “3.6”. In this case, the heart rate in one respiration HR/RR is “1.8” and is less than the threshold.

In a case where the heart rate in one respiration HR/RR is less than the threshold, the display controller 72 is configured to perform control such that the operator can easily recognize that the heart rate in one respiration HR/RR is less than the threshold. For example, the display controller 72 is configured to display an edge of the window W2 in red, on the screen.

In a case where the respiration rate acquisition unit 41 newly receives a notification of the respiratory cycle, the variation rate calculation unit 84 is configured to read out a plurality of stroke volumes esSV calculated in the most recent one respiratory cycle. As described above, in a case where the heart rate in one respiration HR/RR is less than the threshold, the number of pieces of used data read out by the variation rate calculation unit 84 is also less than a threshold. In such a case, the variation rate calculation unit 84 does not calculate the respiratory variation SVV, and stores, for example, an error value indicating an error in the memory 19.

The display controller 72 is configured to read out the error value, as the respiratory variation of the latest hemodynamic parameter stored in the memory 19. Then, for example, as indicated by an arrow A in FIG. 6, the display controller 72 is configured to perform control such that “---” indicating that the respiratory variation SVV of the hemodynamic parameter cannot be calculated is displayed on the screen.

It is assumed that the operator performs an operation of selecting the selection button B21 in such a state. In this case, the numeral displayed in the window W1 is changed from “1” to “2”. The period setting unit 83 increases the number of cycles from “1” to “2”, and sets the predetermined period by using “2”, which is the number of cycles after increase. Then, the period setting unit 83 notifies the variation rate calculation unit 84 of the set predetermined period.

Upon receiving the notification of the predetermined period from the period setting unit 83, the variation rate calculation unit 84 is configured to read out a plurality of stroke volumes esSV calculated in the predetermined period. In a case where the number of pieces of data of the read-out stroke volumes esSV, that is, the number of pieces of used data, is equal to or greater than the threshold, the variation rate calculation unit 84 calculates the respiratory variation SVV by using the plurality of stroke volumes esSV. Accordingly, the stroke volume variation SVV is displayed on the screen.

In a case where the number of pieces of used data is still less than the threshold, the variation rate calculation unit 84 determines that the respiratory variation SVV cannot be calculated, and stores, for example, an error value in the memory 19. In this case, the display indicating that the respiratory variation SVV cannot be calculated is continued on the screen.

### (c) Automatic Mode

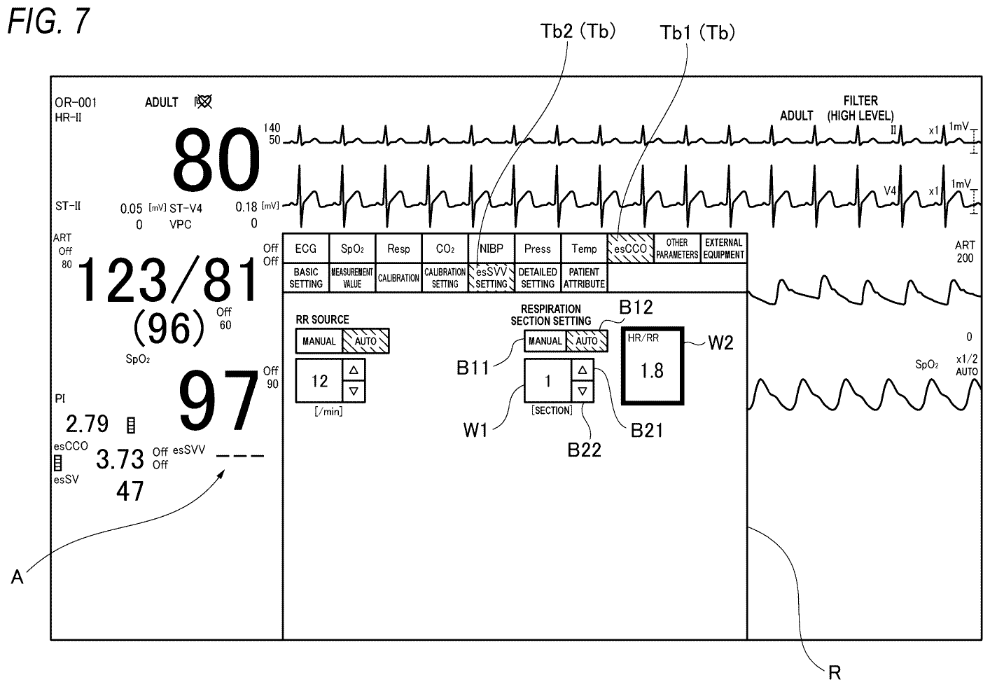

FIGS. 7 to 9 are diagrams for illustrating a state where the length of the predetermined period is automatically changed by the period setting unit 83 illustrated in FIG. 3. FIGS. 7 to 9 illustrate screens displayed in a case where the selection button B12 on the setting screen is selected by the operator.

In a case where the input operation as described above is performed, the acceptance unit 6 illustrated in FIG. 1 is configured to output an instruction signal indicating contents of the input operation to the reception unit 74 of the display device 1. Upon receiving the instruction signal output from the acceptance unit 6, the reception unit 74 outputs the instruction signal to the calculation unit 70.

The hemodynamics calculation unit 17 of the calculation unit 70 is configured to operate in the above-described manual mode, until the instruction signal indicating that the selection button B12 is selected is received. On the other hand, in a case where the instruction signal is received, the hemodynamics calculation unit 17 is configured to operate in the automatic mode.

More specifically, the period setting unit 83 is configured to increase the number of cycles to increase the predetermined period until the number of pieces of used data is equal to or greater than the threshold. Specifically, as illustrated in FIG. 7, it is assumed that the heart rate in one respiration HR/RR displayed in the window W2 is “1.8”. In this case, same as or similarly to the case illustrated in FIG. 6, the variation rate calculation unit 84 notifies the period setting unit 83 that the number of pieces of used data is less than the threshold.

Upon receiving the notification that the number of pieces of used data is less than the threshold, the period setting unit 83 is configured to increase the number of cycles by one to reset the predetermined period, and is configured to notify the variation rate calculation unit 84 and the display controller 72 of the reset predetermined period. The period setting unit 83 is configured to notify the display controller 72 of the number of cycles after increase.

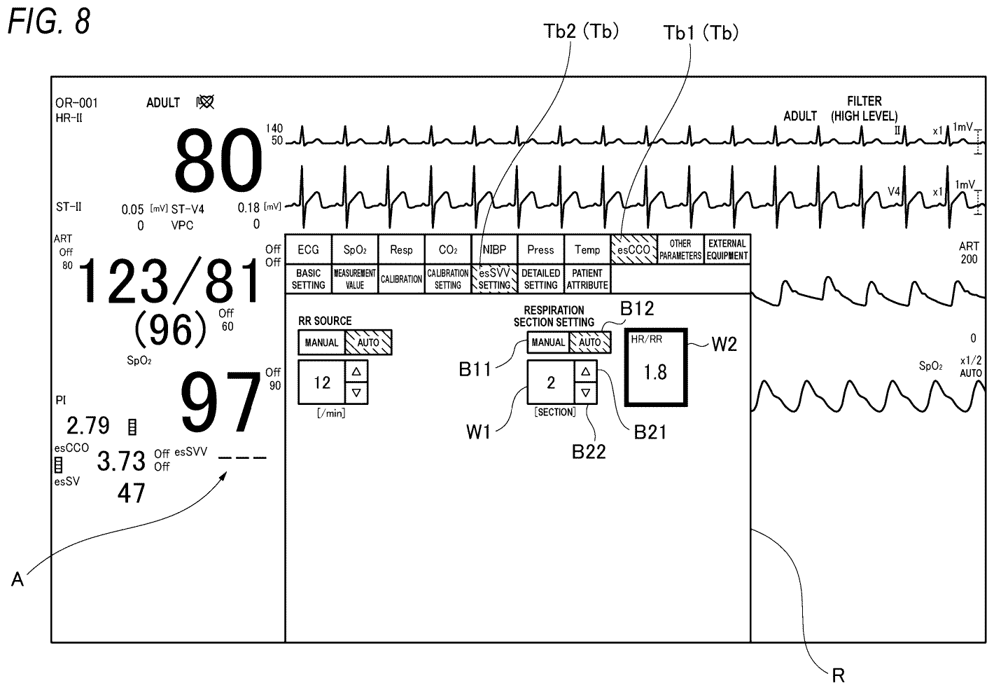

Upon receiving the notification of the number of cycles from the period setting unit 83, the display controller 72 is configured to perform control such that the notified number of cycles is displayed in the window W1. Specifically, the display controller 72 is configured to perform control of changing “1” displayed in the window W1 in FIG. 7 to “2” as illustrated in FIG. 8.

Upon receiving the notification of the predetermined period from the period setting unit 83, the variation rate calculation unit 84 is configured to read out a plurality of stroke volumes esSV calculated in the notified predetermined period and is configured to attempt to calculate the respiratory variation SVV.

Here, it is assumed that the number of pieces of used data included in the two respiratory cycles read out by the variation rate calculation unit 84 is still less than the threshold. In this case, as indicated by an arrow A in FIG. 8, the display indicating that the respiratory variation SVV cannot be calculated is continued on the screen. The variation rate calculation unit 84 again notifies the period setting unit 83 that the number of pieces of used data is less than the threshold.

Upon receiving the notification that the number of pieces of used data is less than the threshold again, the period setting unit 83 increases the number of cycles by one to reset the predetermined period, and notifies the variation rate calculation unit 84 and the display controller 72 of the reset predetermined period. The period setting unit 83 is configured to notify the display controller 72 of the number of cycles after increase.

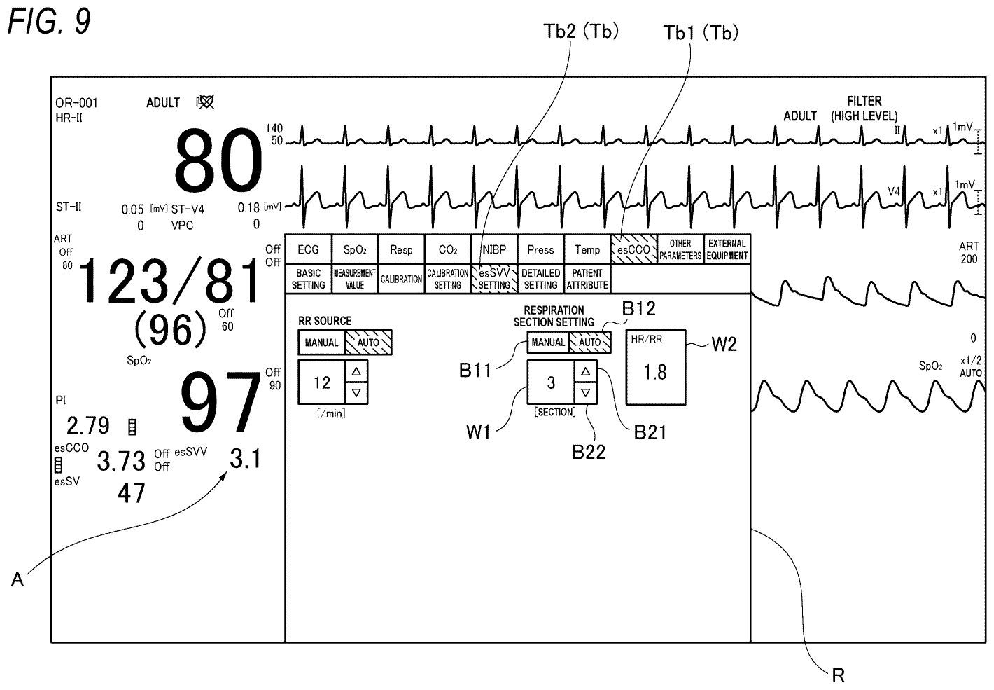

Upon receiving the notification of the number of cycles from the period setting unit 83, the display controller 72 performs control of changing “2” displayed in the window W1 in FIG. 8 to “3” as illustrated in FIG. 9.

Upon receiving the notification of the predetermined period from the period setting unit 83, the variation rate calculation unit 84 is configured to read out a plurality of stroke volumes esSV calculated in the notified predetermined period and is configured to attempt to calculate the respiratory variation SVV. Here, it is assumed that the number of pieces of used data included in the three respiratory cycles read out by the variation rate calculation unit 84 is equal to or greater than the threshold, and the respiratory variation SVV is calculated. In this case, as indicated by an arrow A in FIG. 9, a value of the respiratory variation SVV is, for example, “3.1” displayed on the screen. In this case, for example, the red color of the edge of the window W2 disappears.

### <Procedure of Operation>

### (Outline of Overall Operation)

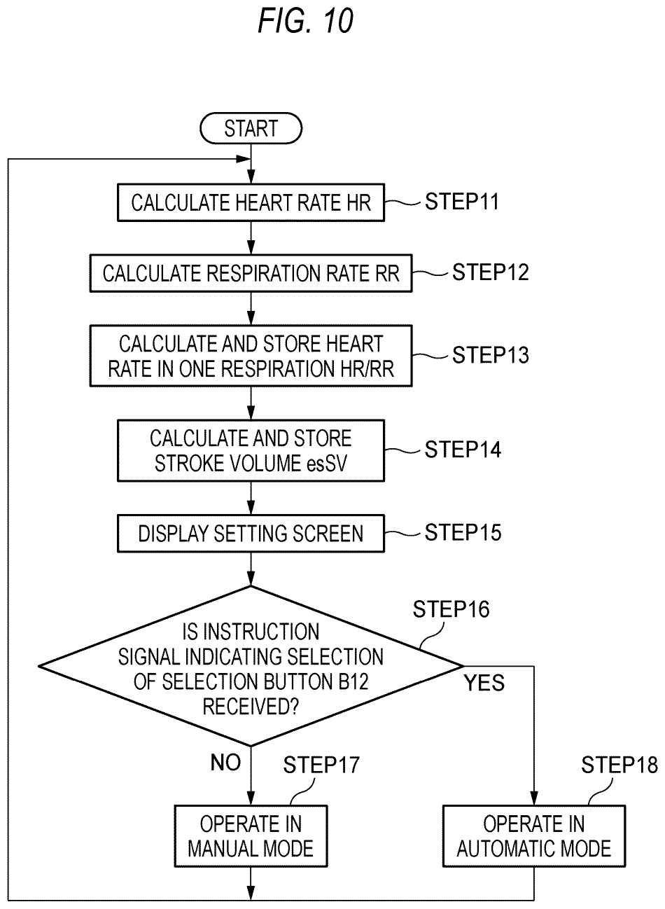

FIG. 10 is a flowchart for illustrating an outline of operations performed when calculating and displaying a respiratory variation of a hemodynamic parameter by the physiological information processing apparatus M according to the embodiment of the present disclosure. Here, a case where the stroke volume variation SVV is calculated and displayed as a respiratory variation of the hemodynamic parameter will be described.

Referring to FIG. 10, first, in a case where the operator activates the physiological information processing apparatus M, each acquisition unit in the calculation unit 70 performs measurement or the like. Specifically, the heart rate acquisition unit 12 is configured to calculate the heart rate HR (STEP 11), and the respiration rate acquisition unit 41 is configured to detect the respiratory cycle and the respiration rate RR (STEP 12).

Next, the HR/RR calculation unit 82 in the hemodynamics calculation unit 17 is configured to calculate the heart rate in one respiration HR/RR, using the heart rate HR calculated in STEP 11 and the respiration rate RR detected in STEP 12. Then, the HR/RR calculation unit 82 is configured to store the calculated heart rate in one respiration HR/RR in the memory 19 (STEP 13).

Next, the parameter calculation unit 81 in the hemodynamics calculation unit 17 is configured to calculate the stroke volume esSV, based on the heart rate HR calculated in STEP 11 or the like, and is configured to store the calculated stroke volume esSV in the memory 19 (STEP 14).

Next, it is assumed that, for example, after a menu button included in a screen is selected and the region R is displayed, the operator further performs an operation of selecting the tab Tb2 for selecting the display of a setting screen in the region R. In this case, the display controller 72 is configured to perform control such that a setting screen as illustrated in FIG. 4 is displayed on the display 71 (STEP 15).

Next, the hemodynamics calculation unit 17 is configured to check whether an instruction signal that indicates that an operation of selecting the selection button B12 is performed by the operator is received (STEP 16). In a case where the instruction signal indicating contents of the operation is not received (“NO” in STEP 16), the hemodynamics calculation unit 17 is configured to operate in the manual mode (STEP 17).

On the other hand, in a case where the instruction signal indicating that the selection button B12 is selected by the operator is received (“YES” in STEP 16), the hemodynamics calculation unit 17 is configured to operate in the automatic mode (STEP 18).

### (Procedure of Operations in Manual Mode)

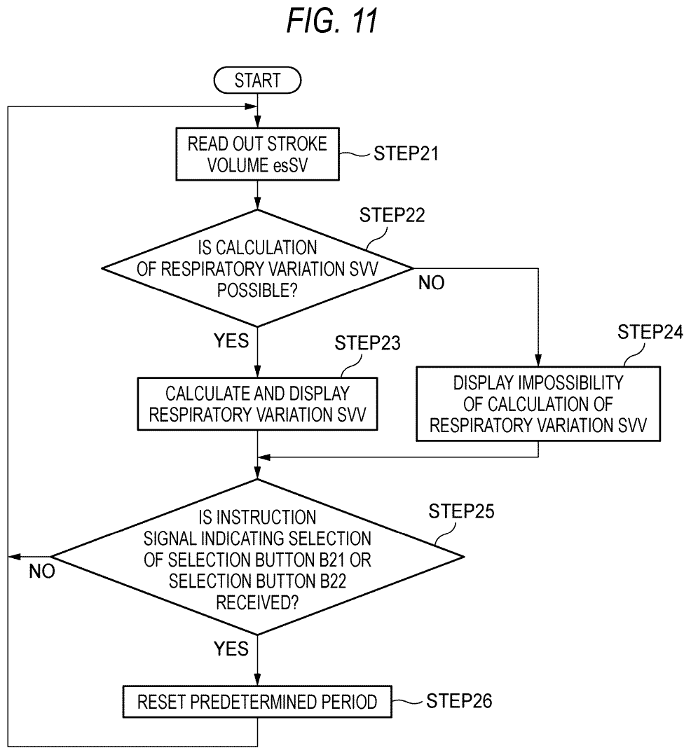

FIG. 11 is a flowchart for illustrating operations in a case where the hemodynamics calculation unit 17 illustrated in FIG. 1 operates in the manual mode.

Referring to FIG. 11, first, upon newly receiving a notification of a respiratory cycle detected by respiration rate acquisition section 41, the variation rate calculation unit 84 is configured to read out a plurality of stroke volumes esSV calculated in a predetermined period based on the notified respiratory cycle. The predetermined period corresponds to the most recent one respiratory cycle (STEP 21).

Next, the variation rate calculation unit 84 is configured to check whether the number of the read-out stroke volumes esSV, that is, the number of pieces of used data, is equal to or greater than a threshold to determine whether the respiratory variation SVV can be calculated (STEP 22).

In a case where the number of pieces of used data is equal to or greater than the threshold, the variation rate calculation unit 84 determines that the respiratory variation SVV can be calculated (“YES” in STEP 22), and calculates the respiratory variation SVV. Then, the display controller 72 is configured to perform control of displaying, on the screen, the respiratory variation SVV calculated by the variation rate calculation unit 84 (STEP 23).

On the other hand, in a case where the number of pieces of used data is less than the threshold, the variation rate calculation unit 84 is configured to determine that the respiratory variation SVV cannot be calculated (“NO” in STEP 22), and is configured to output an error value as the respiratory variation SVV. Then, the display controller 72 is configured to perform control such that, for example, “---” indicating that the respiratory variation of the hemodynamic parameter cannot be calculated is displayed on the screen (STEP 24).

Next, the period setting unit 83 is configured to check whether an instruction signal indicating that an operation of selecting the selection button B21 or the selection button B22 is performed by the operator is received (STEP 25). In a case where the instruction signal indicating contents of the operation is not received (“NO” in STEP 25), the period setting unit 83 does not change the predetermined period, and the operations in STEP 21 and the subsequent steps are performed again.

On the other hand, in a case where the instruction signal indicating that an operation of selecting the selection button B21 or the selection button B22 is performed by the operator is received (“YES” in STEP 25), the period setting unit 83 is configured to change the number of cycles that is the number of respiratory cycles included in the predetermined period, based on the instruction signal and is configured to reset the predetermined period (STEP 26). Then, stroke volumes esSV calculated in the predetermined period after resetting are read out (STEP 21), and the operations in STEP 22 and the subsequent steps are performed again.

### (Procedure of Operations in Automatic Mode)

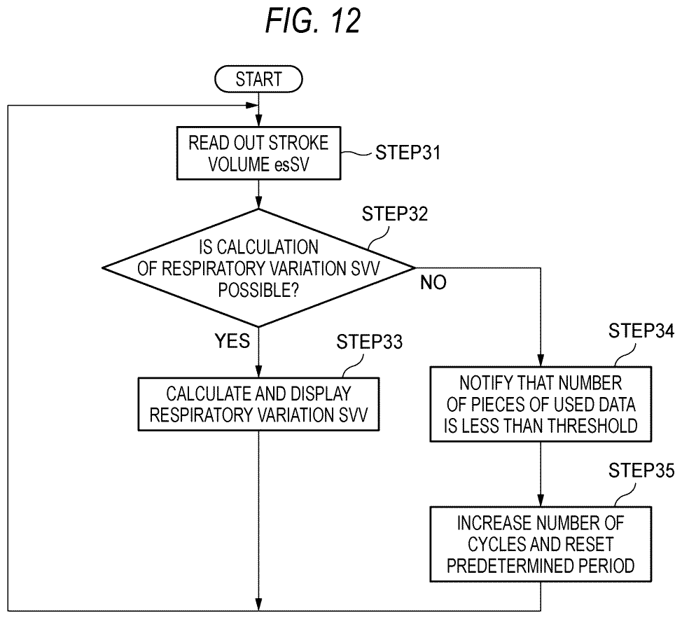

FIG. 12 is a flowchart for illustrating operations in a case where the hemodynamics calculation unit 17 illustrated in FIG. 1 operates in the automatic mode.

Since operations from STEP 31 to STEP 33 illustrated in FIG. 12 are the same as or similar to the operations from STEP 21 to STEP 23 illustrated in FIG. 11, a detailed description thereof will not be repeated here. In a case where the respiratory variation SVV is calculated and displayed (STEP 33), the operations in STEP 31 and the subsequent steps are performed again.

In STEP 32, in a case where the number of pieces of used data is less than the threshold, the variation rate calculation unit 84 determines that the respiratory variation SVV cannot be calculated (“NO” in STEP 32). In this case, the variation rate calculation unit 84 notifies the period setting unit 83 that the number of pieces of used data is less than the threshold (STEP 34).

Next, upon receiving the notification of the above contents from the variation rate calculation unit 84, the period setting unit 83 increases the number of cycles by one and resets the predetermined period (STEP 35). Then, stroke volumes esSV calculated in the predetermined period after the resetting is read out (STEP 31), and the operations in STEP 32 and the subsequent steps are performed again.

In a case of operating in either the manual mode or the automatic mode, for example, in a case where a predetermined period of time elapses from a timing at which the number of cycles is increased, the hemodynamics calculation unit 17 returns the number of cycles to “1”. Accordingly, the number of cycles can be prevented from continuously increasing. An upper limit of the number of cycles is, for example, “8”.

As described above, in the physiological information processing apparatus M according to an aspect of the present disclosure, the heart rate acquisition unit 12 is configured to acquire the heart rate HR of a subject. The respiration rate acquisition unit 41 is configured to acquire the respiration rate RR of the subject. The hemodynamics calculation unit 17 is configured to calculate a hemodynamic parameter of the subject, and is configured to calculate the respiratory variations SVV and PPV of the hemodynamic parameter based on a plurality of hemodynamic parameters calculated in a predetermined period. The display controller 72 is configured to output the calculated respiratory variations SVV and PPV to the display 71. The hemodynamics calculation unit 17 is further configured to calculate the heart rate in one respiration HR/RR, which is a heart rate included in one respiratory cycle, using the heart rate HR and the respiration rate RR. The display controller 72 is further configured to output, to the display 71, a setting screen for setting the length of the predetermined period, the setting screen including a screen configured to display the heart rate in one respiration HR/RR. Then, the hemodynamics calculation unit 17 is configured to acquire setting information indicating contents set on the setting screen, is configured to set a length of the predetermined period based on the acquired setting information, and is configured to calculate the respiratory variations SVV and PPV in the predetermined period after the setting.

With such a configuration, for example, the operator can determine whether the number of pieces of data of the hemodynamic parameters used to calculate the respiratory variations SVV and PPV, that is, the number of pieces of data of the hemodynamic parameters calculated in the predetermined period, is sufficient by checking the heart rate in one respiration HR/RR displayed on the display 71. In a case where it is determined that the number of pieces of data is not sufficient, the operator can set the predetermined period to be long on the setting screen so as to increase the number of pieces of data. Accordingly, the respiratory variations SVV and PPV of the hemodynamic parameter can be calculated using a sufficient number of pieces of data, and thus a state of the subject can be monitored more accurately.

In the physiological information processing apparatus M according to another aspect of the present disclosure, the length of the predetermined period is a length of one or more respiratory cycles. The display controller 72 is configured to cause the display to display the setting screen in which the number of respiratory cycles included in the predetermined period is configured to be set. The hemodynamics calculation unit 17 is configured to set a length of the predetermined period based on a respiratory cycle, which is detected based on at least one of a start timing and an end timing of respiration of the subject, and based on the number of respiratory cycles set on the setting screen. As described above, with the configuration in which the number of respiratory cycles can be set on the setting screen, the length of the predetermined period can be easily changed by the operator.

In the physiological information processing apparatus M according to another aspect of the present disclosure, the respiration rate acquisition unit 41 is further configured to determine at least one of the start timing and the end timing of respiration of the subject, based on a detection result from the respiration measurement device 4 that detects a concentration of carbon dioxide in expired air of the subject.

In general, many of the cardiac output measuring devices have a function of measuring the stroke volume variation SVV, and are specialized in measuring a cardiac output and a related parameter. In these devices, a function of measuring the respiratory cycle is not provided, and the respiratory cycle is set as a fixed value. In contrast, in the physiological information processing apparatus M according to the presently disclosed subject matter, with the above-described configuration, the respiratory cycle can be more accurately determined, and thus the respiratory variations SVV and PPV of the hemodynamic parameter can be more accurately calculated.

In the physiological information processing apparatus M according to another aspect of the present disclosure, the display controller 72 is further configured to cause the display to display the setting screen in which automatic change of the length of the predetermined period is configured to be set. In a case where the automatic change of the length of the predetermined period is set on the setting screen and the number of pieces of used data of the hemodynamic parameter calculated in the predetermined period is less than a threshold, the hemodynamics calculation unit 17 is configured to set the predetermined period to be long. As described, with the configuration in which the length of the predetermined period is automatically changed according to the number of pieces of used data of the hemodynamic parameter calculated in the predetermined period, it is possible to reduce the time and labor of the operator.

In the physiological information processing apparatus M according to another aspect of the present disclosure, when performing the automatic change of the length of the predetermined period, the hemodynamics calculation unit 17 increases the number of respiratory cycles included in the predetermined period until the number of pieces of used data becomes equal to or greater than the threshold, and sets the length of the predetermined period based on a respiratory cycle, which is detected based on at least one of a start timing and an end timing of respiration of the subject, and based on the number of respiratory cycles included in the predetermined period.

As described above, with the configuration in which the number of respiratory cycles included in the predetermined period is increased when the number of pieces of data of the hemodynamic parameter calculated in the predetermined period is less than the threshold, and the increase in the number of the respiratory cycles included in the predetermined period is ended when the number of pieces of data is equal to or greater than the threshold, the number of pieces of data used to calculate the respiratory variations SVV and PPV of the hemodynamic parameter can be set to an appropriate number.

In the physiological information processing apparatus M according to another aspect of the present disclosure, the hemodynamics calculation unit 17 does not calculate the respiratory variations SVV and PPV in a case where the number of pieces of data of the hemodynamic parameter calculated in the predetermined period is less than a threshold. With such a configuration, since it is possible to avoid calculation and display of the respiratory variations SVV and PPV having low accuracy, the operator can more accurately monitor the physiological information of the subject.

Although the embodiments of the present disclosure have been described above, the technical scope of the present application should not be construed as being limited to the description of the embodiments. The embodiments are merely an example, and it is understood by those skilled in the art that various modifications of the embodiments are possible within the scope of the inventions described in the claims. The technical scope of the present application should be determined based on the scope of the inventions described in the claims and equivalents thereof.

This application claims priority to Japanese Patent Application No. 2022-127788 filed on Aug. 10, 2022, the entire content of which is incorporated herein by reference.

### INDUSTRIAL APPLICABILITY

According to the presently disclosed subject matter, it is possible to provide a physiological information display apparatus and a physiological information display method capable of more accurately monitoring a state of a subject.

1. A physiological information display apparatus comprising:
a heart rate acquisition unit configured to acquire a heart rate of a subject;
a respiration rate acquisition unit configured to acquire a respiration rate of the subject;
one or more processors configured to calculate:
a hemodynamic parameter of the subject; and
a respiratory variation of the hemodynamic parameter, based on a plurality of the hemodynamic parameters calculated in a predetermined period; and
a display controller configured to output the respiratory variation to a display,
wherein the one or more processors is further configured to calculate a heart rate in one respiration, using the heart rate and the respiration rate, the heart rate in one respiration being a heart rate included in one respiratory cycle,
the display controller is further configured to output, to the display, a setting screen for setting a length of the predetermined period, the setting screen configured to display the heart rate in one respiration, and
the one or more processors configured to:
acquire setting information indicating contents set on the setting screen;
set the length of the predetermined period based on the acquired setting information; and
calculate the respiratory variation in the set predetermined period.

2. The physiological information display apparatus according to claim 1,
wherein the length of the predetermined period is a length of one or more respiratory cycles,
the display controller is configured to cause the display to display the setting screen in which the number of respiratory cycles included in the predetermined period is configured to be set, and
the one or more processors is configured to set the length of the predetermined period, based on a respiratory cycle and the number of respiratory cycles, the respiratory cycle being detected based on at least one of a start timing and an end timing of respiration of the subject, the number of respiratory cycles being set on the setting screen.

3. The physiological information display apparatus according to claim 2,
wherein the respiration rate acquisition unit is further configured to determine at least one of the start timing and the end timing of respiration of the subject, based on a detection result from a detection device, the detection device being configured to detect a concentration of carbon dioxide in expired air of the subject.

4. The physiological information display apparatus according to claim 1,
wherein the display controller is further configured to cause the display to display the setting screen in which automatic change of the length of the predetermined period is configured to be set, and
the one or more processors is configured to set the predetermined period to be long, in a case where the automatic change of the length of the predetermined period is set on the setting screen and the number of pieces of data of the hemodynamic parameter calculated in the predetermined period is less than a threshold.

5. The physiological information display apparatus according to claim 4,
wherein the length of the predetermined period is a length of one or more respiratory cycles, and
in a case where the one or more processors performs the automatic change of the length of the predetermined period, the one or more processors is configured to:
increases the number of respiratory cycles included in the predetermined period until the number of pieces of data becomes equal to or greater than the threshold; and
set the length of the predetermined period, based on a respiratory cycle and the number of respiratory cycles, the respiratory cycle being detected based on at least one of a start timing and an end timing of respiration of the subject, respiratory cycles being included in the predetermined period.

6. The physiological information display apparatus according to claim 1,
wherein the one or more processors is configured not to calculate the respiratory variation, in a case where the number of pieces of data of the hemodynamic parameter calculated in the predetermined period is less than a threshold.

7. A physiological information display method comprising:
acquiring a heart rate of a subject;
acquiring a respiration rate of the subject;
calculating a heart rate in one respiration using the heart rate and the respiration rate, the heart rate in one respiration being a heart rate included in one respiratory cycle;
calculating a hemodynamic parameter of the subject;
outputting, to a display, a setting screen for setting a length of a predetermined period, the setting screen configured to display the heart rate in one respiration;
setting the length of the predetermined period based on contents set on the setting screen;
calculating a respiratory variation of the hemodynamic parameter, based on a plurality of the hemodynamic parameters calculated in the set predetermined period; and
outputting the respiratory variation to the display.
